(19) United States
(12) Patent Application Publication
(10) Pub. No.: US 2026/0060661 A1
(43) Pub. Date: Mar. 5, 2026

(54) MICROSURGICAL-ROBOT POWER MECHANISM AND SETTING METHOD FOR INSTRUMENT ORIGIN INITIALIZATION THEREOF

(71) Applicant: Koutech Medical Robotics (Shanghai) Co., Ltd, Shanghai (CN)

(72) Inventor: Kou Watanabe, Shanghai (CN)

(21) Appl. No.: 19/209,983

(22) Filed: May 16, 2025

(30) Foreign Application Priority Data
Aug. 27, 2024 (CN) 202411183112.5

(51) Int. Cl. A61B 17/00; A61B 34/30; A61B 90/00; G16H 40/63

(52) U.S. Cl. CPC A61B 17/00234; A61B 34/30; A61B 90/03; G16H 40/63; A61B 2017/00345; A61B 2017/00398; A61B 2017/00477; A61B 2090/035

(57) ABSTRACT

Provided are a microsurgical-robot power mechanism and a setting method for instrument origin initialization thereof. The power mechanism includes a plurality of drive motors, a plurality of motor turntables, intermediate turntables and connecting turntables, where an output shaft of each of the drive motors is connected to the motor turntable, and the motor turntable is connected to the connecting turntable through the intermediate turntable. The connecting turntable is provided with a limiting protrusion mated with a stopper on a limiting support bracket, and when rotated to a preset angle, the connecting turntable is in contact with the limiting protrusion so as to block rotation. The power mechanism is further provided with an engagement determination mechanism configured to determine an engagement status of the connecting turntable with the motor turntable or the intermediate turntable.

### CROSS REFERENCE TO RELATED APPLICATION

The present disclosure claims the priority to the Chinese patent application with the filing No. 2024111831125 filed with the Chinese Patent Office on Aug. 27, 2024, and entitled “MICROSURGICAL-ROBOT POWER MECHANISM AND SETTING METHOD FOR INSTRUMENT ORIGIN INITIALIZATION THEREOF”, which is incorporated herein by reference in entirety.

### TECHNICAL FIELD

The present disclosure relates to the field of medical instruments, and specifically to a microsurgical-robot power mechanism and a setting method (calibration method) for instrument origin initialization thereof.

### BACKGROUND ART

In the field of microsurgical instruments, due to the small size of the instruments, they require extremely high operational precision. Under such circumstances, conventional technical solutions of determining a mechanical engagement status by torque, current and speed faces significant challenges. Specifically, these conventional methods require very high selection of determination threshold, and if the threshold is not properly set, false determination is likely to occur. This is especially prominent in microsurgical scenarios because the instruments are extremely small in size and a force required for rotation thereof is quite small, it is almost difficult for a conventional torque or current detection method to achieve accurate initial position determination. This challenge imposes substantial constraints on the application of robotic technologies in the field of microsurgery.

In the prior art, CN106102641A and CN107660139A propose determining whether an instrument or a sterile adapter has reached a stopper by identifying a motor torque, so as to determine whether the instrument or the sterile adapter is engaged with a motor. Although this solution is effective under certain scenarios, significant restrictions still exist in microsurgical instruments due to constraints imposed by instrument dimensions and operating force values.

Further, CN113164211A attempts to determine an engagement status between an instrument and a motor by determining whether the instrument is physically constrained by identifying motor torque, current, speed or their combination. Although this method expands detection dimensionality, in the field of microsurgical instruments, due to tiny force values of the instruments, it still may face a problem of false determination caused by improper threshold selection.

In addition, CN114929150A proposes a method independent of a physical constraint, where through mutual resistance between multiple motor controllers, an engagement status between an instrument and motors is determined by identifying currents and speeds of motors, and a position where an instrument is located is determined through angles of rotation of the motors. This solution attempts to evade shortcomings of the conventional method, but its complexity and requirement on system precision make it still challenging in practical application.

To sum up, although the prior art has proposed some solutions to solve the problem of mechanical engagement determination, in the field of microsurgical instruments, due to constraints of micro-scale dimensions and operating force values of the instruments, these solutions still have major shortcomings. The development of microsurgical robots urgently requires an innovative technology capable of accurately identifying the instrument engagement status, independent of the conventional torque or current determination method.

### SUMMARY

In order to solve the above technical problem, the present disclosure provides a microsurgical-robot power mechanism and a setting method for instrument origin initialization thereof.

The present disclosure is implemented through following technical solutions.

A microsurgical-robot power mechanism includes drive motors, motor turntables, intermediate turntables and connecting turntables, where several drive motors are provided, an output shaft of each of the several drive motors is respectively assembled and connected to one motor turntable; and the motor turntable is assembled and connected to the connecting turntable through the intermediate turntable, or is directly assembled and connected to the connecting turntable;
the several connecting turntables are provided in a limiting support bracket, each of the connecting turntables is provided with a limiting protrusion on a peripheral surface, the limiting support bracket is provided with a stopper, and when rotated to a certain angle, the connecting turntable is in contact with the limiting protrusion, such that rotation of the connecting turntable is blocked;
each of the several connecting turntables is respectively assembled and connected to a driven member, and types of the driven member include instrument and sterile adapter; and
the power mechanism is provided with an engagement determination mechanism, and configured to determine an engagement status of the connecting turntable with the motor turntable or the intermediate turntable.

Further, the output shaft of each of the drive motors is mounted with an encoder, where the encoder is an absolute motor encoder.

Further, the connecting turntables are disc-shaped members, with one end having several tooth-shaped protrusions in a circular array, and the other end connected to the driven member; the intermediate turntables are cylindrical members with two ends respectively having several tooth-shaped protrusions in a circular array, both ends being connected to the connecting turntable and the motor turntable; the motor turntables are rod-shaped members, with one end having several tooth-shaped protrusions in a circular array, and the other end connected to the drive motor; and tips of the tooth-shaped protrusions on the motor turntables, the intermediate turntables and the connecting turntables are provided with rounded or chamfered transitions.

Further, bodies of the several drive motors are simultaneously fixedly connected to a motor support bracket; the motor turntables, the intermediate turntables, and the connecting turntable are all located in positions between the limiting support bracket and the motor support bracket; and the limiting support bracket and the motor support bracket are provided with a guiding support bracket, where relative position of the guiding support bracket and the motor support bracket is fixed; and
the limiting support bracket has a trend of continuously moving towards the motor support bracket under an external force; the engagement determination mechanism is provided between the limiting support bracket and the motor support bracket, and when the connecting turntable is engaged with the motor turntable or the intermediate turntable, the limiting support bracket drives the engagement determination mechanism to move, and the engagement determination mechanism sends a signal, and conversely, no signal is sent.

Further, the limiting support bracket is provided with several turntable slots assembled and connected to the connecting turntables, where the turntable slots are circular blind slots, a center of circle of the blind slots is provided with a through hole configured to connect the driven member, and the several turntable slots directly communicate with each other or are independent from each other; and
an outer side of each of the turntable slots is provided with an arc-shaped limiting recess mated with the limiting protrusion, where the limiting protrusion is inserted into the arc-shaped limiting recess; and the arc-shaped limiting recess communicates with the turntable slot, and has an inside diameter equal to an outside diameter of the turntable slot, and an outer edge profile being arc-shaped.

Further, the engagement determination mechanism includes a press rod, a reset spring and a position sensor, where the press rod has one end fitted on the limiting support bracket all the time under an effect of the reset spring, and the other end penetrating through the guiding support bracket to be assembled and connected to the position sensor, and when the connecting turntable is engaged with the motor turntable or the intermediate turntable, the position sensor is triggered, and conversely, the position sensor is not triggered; the reset spring has one end fixed on the position sensor, and the other end fitted on a shoulder of the press rod; and the position sensor is a photoelectric sensor or a Hall sensor or a contact sensor (such as micro-switch).

A structural design of the present disclosure takes the drive motors as a core, the drive motors are connected to the motor turntables via the output shafts, and then the motor turntables are assembled and connected to the connecting turntables through the intermediate turntables or directly. Such multi-level connection method enables more flexible and accurate power transmission, and is adapted to requirements for micro-scale displacement and multi-degree-of-freedom control of a microsurgical robot in surgical operations. Configuration of multiple drive motors can achieve multi-directional and multi-dimensional motion control, so as to meet complicated operation requirements of microsurgery.

A design of the limiting support bracket plays a critical role in this technical solution, and particularly through cooperation of the limiting protrusions with the stoppers, the connecting turntables are effectively prevented from further rotating when reaching a specific angle. This stopper mechanism not only safeguards the drive motors and the transmission mechanism, but also ensures that the robot will not misoperate due to excessive rotation during operation. In addition, through rational design of the limiting support bracket, the whole power mechanism reduces wear of moving parts while ensuring precision, thereby elevating stability and service life of the system.

Configuration of the engagement determination mechanism further enhances an intelligent level of the power mechanism. The engagement determination mechanism can accurately determine the engagement status of the connecting turntable with the motor turntable or the intermediate turntable, which is critical to ensure reliability of power transmission. Through a signal sent by the engagement determination mechanism, a control system can monitor the engagement status in real time, and ensure each action to be performed in a controlled state, thereby improving safety and precision of the system.

The output shaft of each drive motor is mounted with an absolute motor encoder or an incremental motor encoder, which design ensures that even when the motor is powered off or subjected to external interference, the system can still maintain accurate position information. Application of such encoders enables higher precision and reliability of the power mechanism, and helps improve performance of the microsurgical robot in actual operations.

A setting method for instrument origin initialization of a microsurgical robot in the present disclosure includes steps of:
assembling a driven member with a motor turntable or an intermediate turntable through a connecting turntable, where
when a control system identifies an assembling action of the driven member, the connecting turntable is in contact with the motor turntable or the intermediate turntable but is not fully engaged in this case;
then operating, by the control system, the drive motor to rotate, until the connecting turntable is engaged with the motor turntable or the intermediate turntable under an external force;
once the control system identifying engagement of the connecting turntable with the motor turntable or the intermediate turntable, continuing to operate the drive motor to rotate, until the connecting turntable is rotated to a preset stopper on a limiting support bracket and is blocked from rotating;
reading, by the control system, an output of an encoder in real time, where when a position signal output by the encoder remains unchanged, the control system determines that a shaft of the drive motor is restricted, and a status in which the connecting turntable is rotated to the preset stopper and is blocked from rotating is identified; and
storing an absolute position encoding at this instant as an origin in the control system, thus completing setting of origin initialization.

Further, the control system identifies the assembling action of the driven member by a sensor, where the sensor is in communication connection with the control system.

Further, the control system identifies engagement of the connecting turntable 4 with the motor turntable 2 or the intermediate turntable 3 by the engagement determination mechanism 8 in communication connection with the control system.

The setting method for instrument origin initialization is an important function in a microsurgical robot system. This method can ensure that the robot achieves accurate positioning prior to each operation, thereby realizing high-precision surgical operations. The method first assembles the driven member with the motor turntable or the intermediate turntable through the connecting turntable. At this stage, the system can identify the assembling action of the driven member by the sensor, thus ensuring smooth progress of the assembly process.

During engagement, the drive motor starts to rotate, so that the connecting turntable is gradually engaged with the motor turntable or the intermediate turntable under an external force. In this case, the engagement determination mechanism plays a critical role, and is capable of accurately identifying the engagement status and transmitting information to the control system, so that the system monitors the whole engagement process in real time.

When the connecting turntable is rotated to a position of the stopper and is blocked from rotating, the control system can determine whether a current shaft position is affected by the stopper by reading an output of the encoder in real time, and at the same time, the control system monitors current data of the motor as auxiliary parameter. When a position signal output by the encoder remains unchanged, it is indicated that the shaft of the drive motor is restricted, and the system can store an absolute position encoding at this instant as the origin, thus completing the setting of the origin initialization.

The origin calibrated by this method can effectively avoid operational errors caused by displacement of the origin during operation. The setting method streamlines an initialization workflow while ensuring precision, and improves use convenience and operational safety of the system.

Beneficial effects of the present disclosure are as follows.

Improved origin initialization precision: by providing the stopper as the origin of the instrument, the potential problem of false determination in the microsurgical instrument by the conventional torque, current or speed detection method is effectively evaded, a stable and repeatable reference point is provided, thereby enhancing accuracy and reliability of the origin initialization.

Verification of instrument engagement status: the engagement determination mechanism is capable of accurately determining the engagement status between the connecting turntable and the motor turntable after instrument assembly, thus ensuring proper installation of the instrument, thereby reducing operational errors caused by undesirable engagement.

Adaptation to high-precision operation requirements: the precision problem caused by micro-scale dimensions and small operating force values of the microsurgical instrument is effectively solved, thus elevating application performance of the system in high-precision surgical environments.

### BRIEF DESCRIPTION OF DRAWINGS

FIG. 1: a perspective structural schematic view of the present disclosure;

FIG. 2: another perspective structural schematic view of the present disclosure;

FIG. 3: a perspective structural cross-sectional view of the present disclosure;

FIG. 4: another perspective structural cross-sectional view of the present disclosure;

FIG. 5: is a bottom view of a limiting support bracket in the present disclosure;

FIG. 6: a perspective structural schematic view of a connecting turntable in the present disclosure;

FIG. 7: a bottom view of the connecting turntable in the present disclosure;

FIG. 8: a perspective structural schematic view of a motor support bracket and engagement determination mechanisms in the present disclosure;

FIG. 9: a perspective structural schematic view of the motor turntable, an intermediate turntable and the connecting turntable in the present disclosure;

FIG. 10: a schematic view of a status of the engagement determination mechanisms when triggered in the present disclosure;

FIG. 11: a schematic view of a status of the connecting turntables when reaching stoppers in the present disclosure;

FIG. 12: another schematic view of the status of the connecting turntables when reaching the stoppers in the present disclosure; and

FIG. 13: a schematic view of another arrangement form of arc-shaped limiting recesses in the present disclosure.

In the drawings: 1—drive motor, 2—motor turntable, 3—intermediate turntable, 4—connecting turntable, 5—limiting support bracket, 6—guiding support bracket, 7—motor support bracket, 8—engagement determination mechanism, 41—limiting protrusion, 51—turntable slot, 52—arc-shaped limiting recess, 81—press rod, 82—reset spring, 83—position sensor.

### DETAILED DESCRIPTION OF EMBODIMENTS

Below the present disclosure is further described in conjunction with drawings and embodiments.

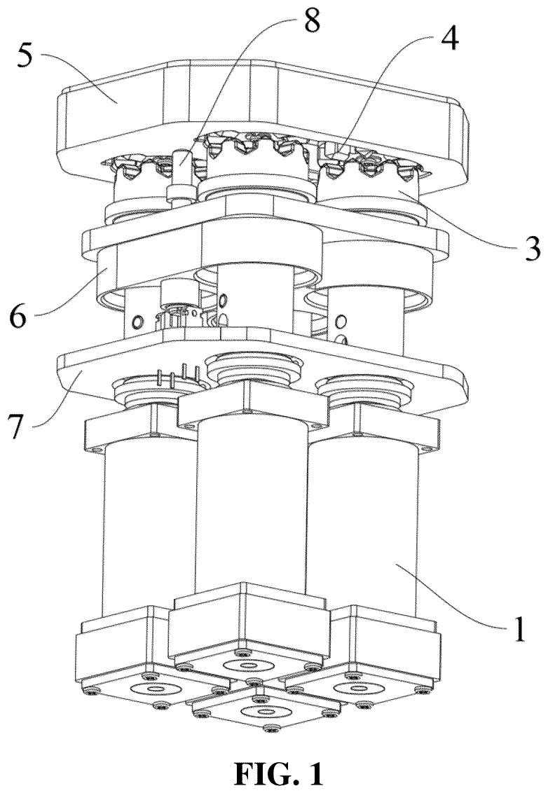

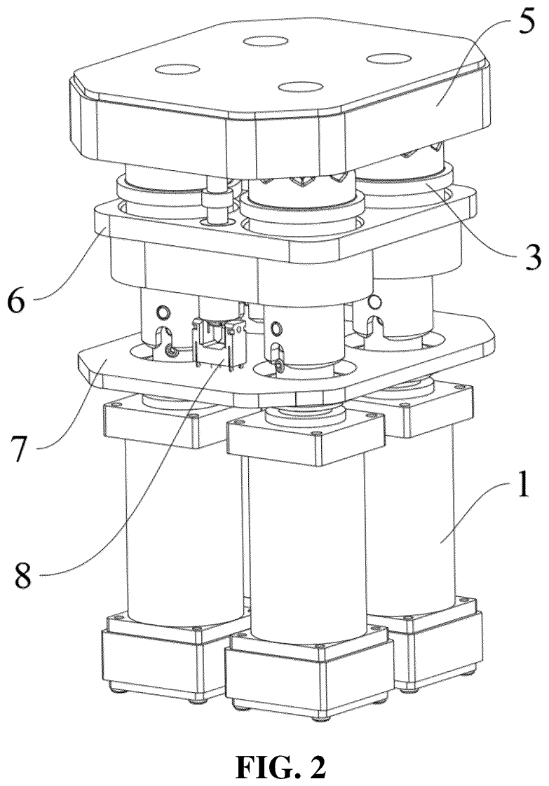

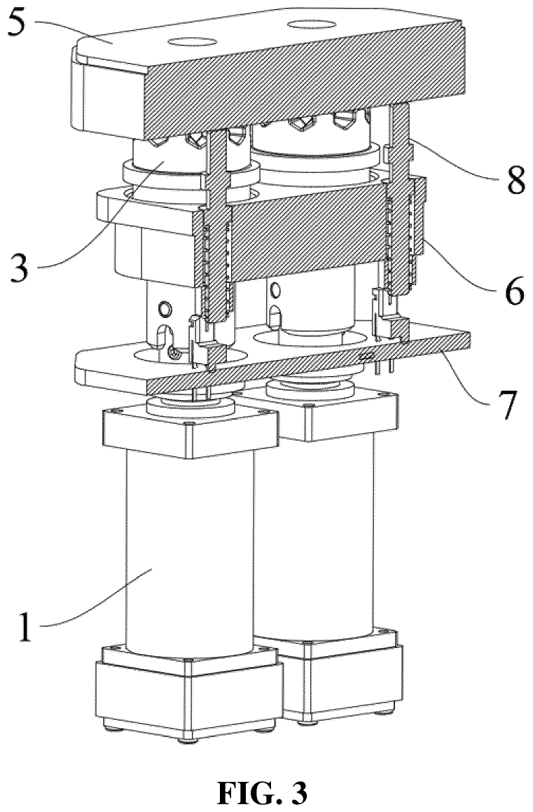

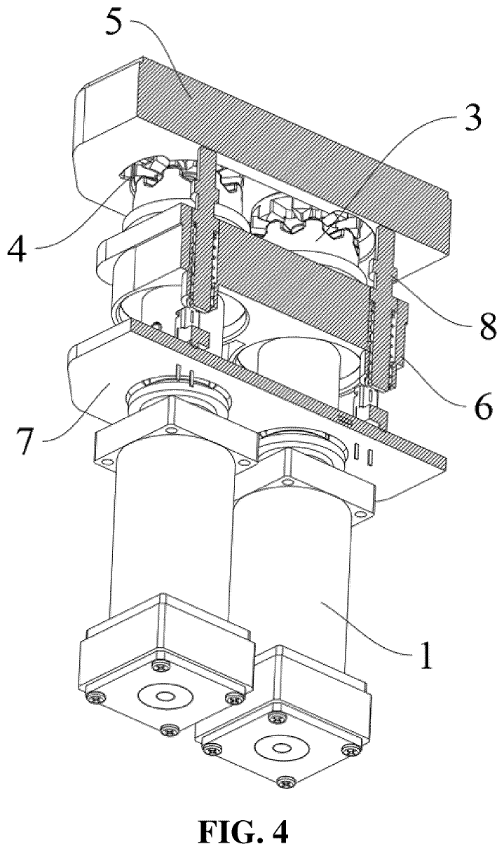

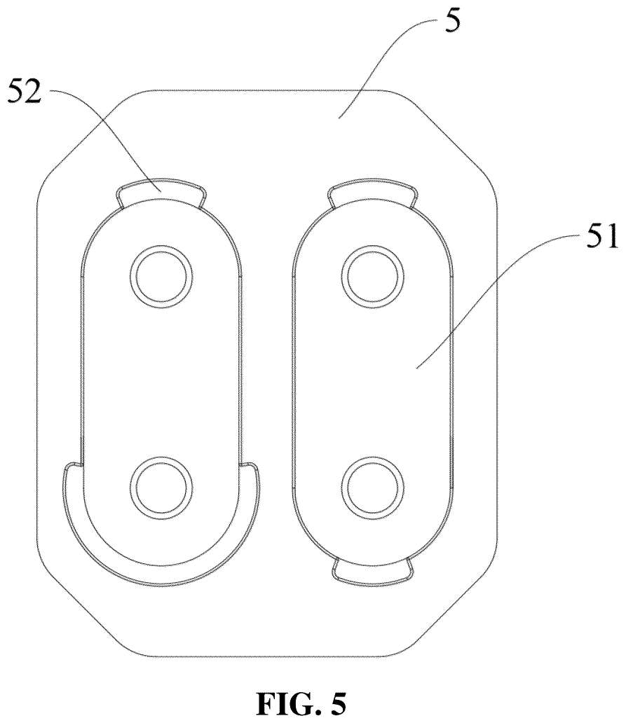

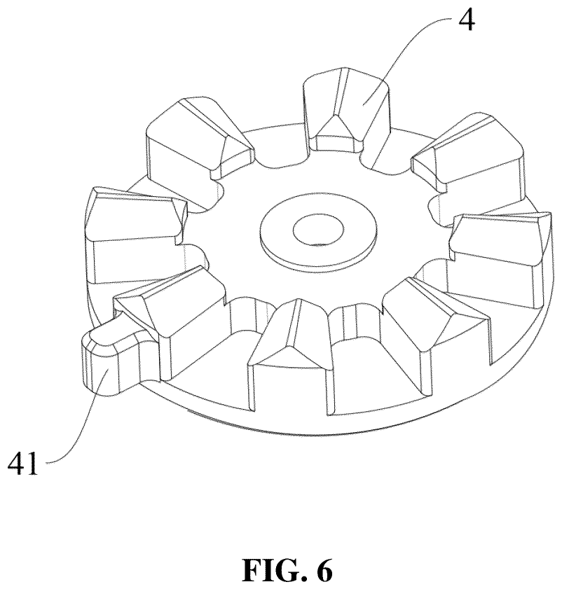

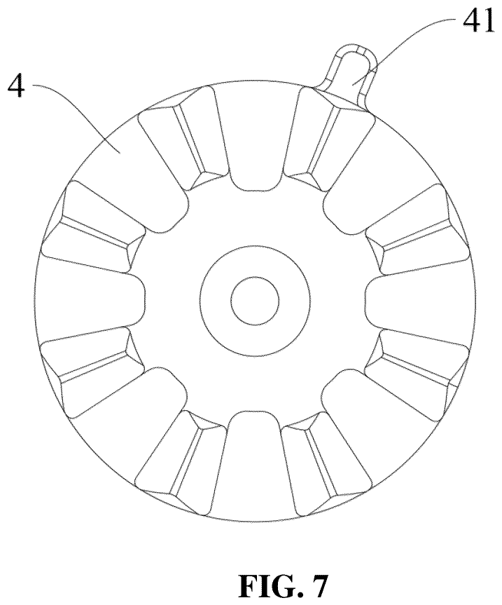

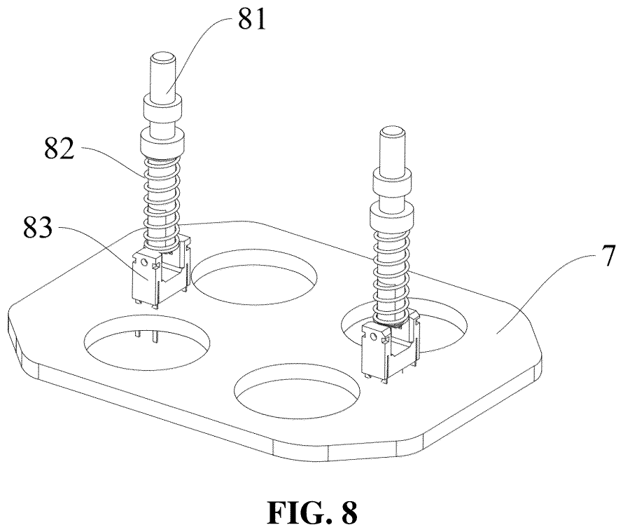

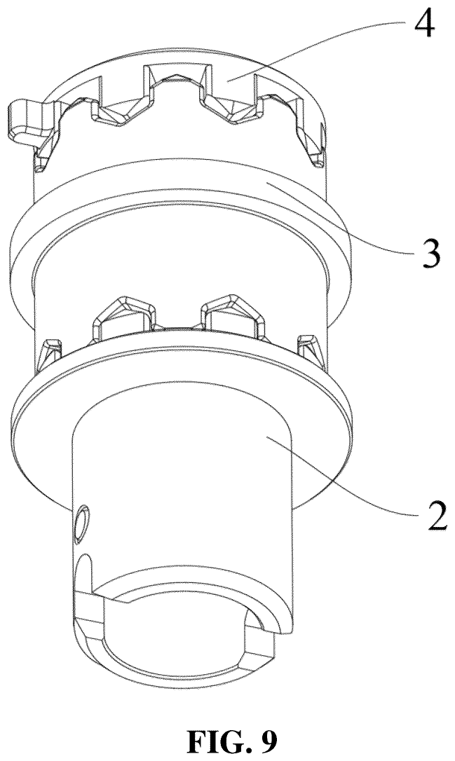

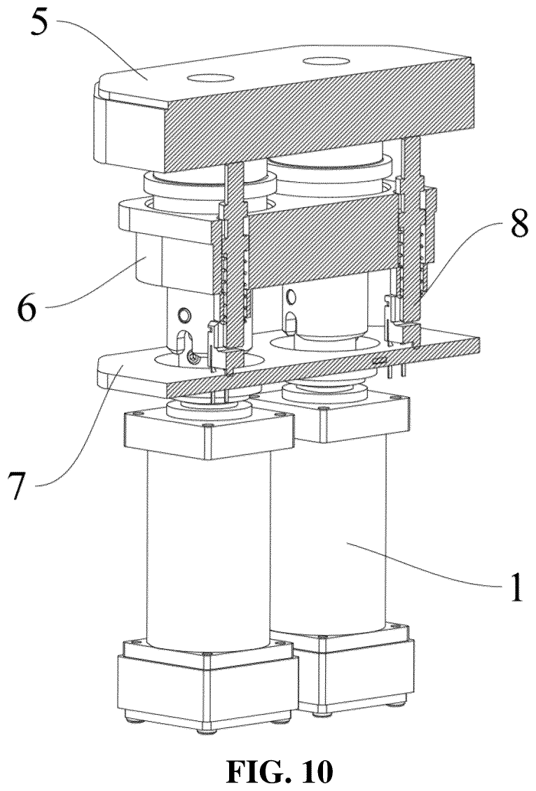

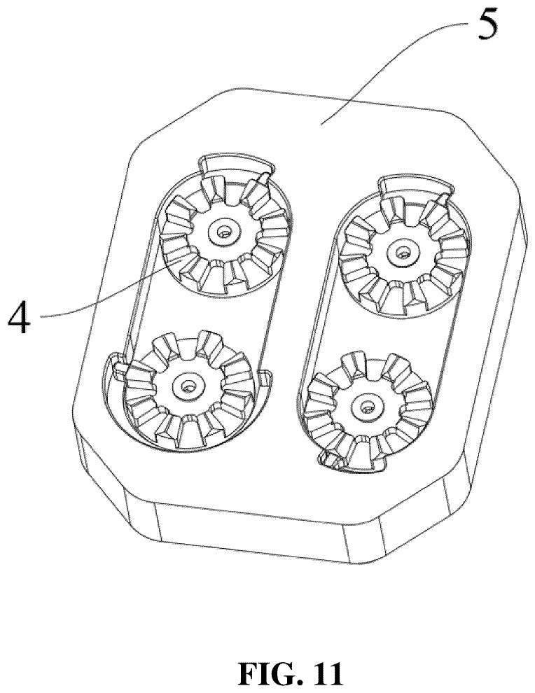

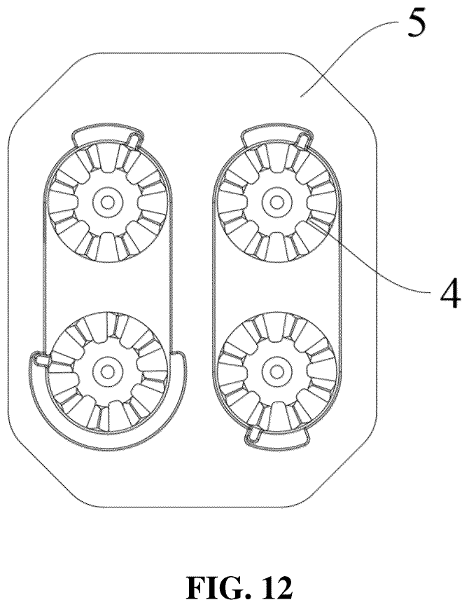

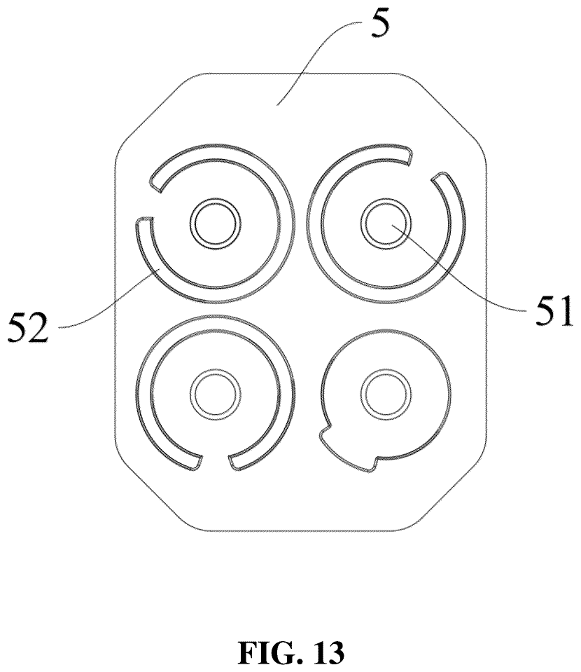

Embodiments: as shown in FIGS. 1-13, a microsurgical-robot power mechanism includes drive motors 1, motor turntables 2, intermediate turntables 3 and connecting turntables 4. A plurality of drive motors 1 are connected to the motor turntables 2 via output shafts, and the motor turntables 2 are meshed with the connecting turntables 4 through the intermediate turntables 3 or directly. The connecting turntables 4 are provided in a limiting support bracket 5, and provided with limiting protrusions 41 at peripheries thereof, so as to realize physical limitation with arc-shaped limiting recesses 52 on the limiting support bracket 5. A driven member (instrument/sterile adapter) is fitted through a connecting port at a tail end of the connecting turntables 4.

Critical structural features are as follows:
transmission assemblies: the motor turntables 2, the intermediate turntables 3 and the connecting turntables 4 are all provided with tooth-shaped protrusions in a circular array, where tooth tips are rounded so as to reduce meshing impact;
limiting mechanism: an arc length of the arc-shaped limiting recesses 52 of the limiting support bracket 5 determines a maximum angle of rotation of the connecting turntables 4, corresponding to a travel range of the instrument;
engagement detection: an engagement determination mechanism 8 is composed of a press rod 81, a reset spring 82 and a position sensor 83 (photoelectric/Hall/micro-switch), and when the connecting turntable 4 is fully meshed with the motor turntable 2, the limiting support bracket 5 is compressed and triggers a sensor signal; and
encoder configuration: an output shaft of each of the drive motors 1 is integrated with an absolute encoder, and position information is still retained after power-off.

A structural design of the microsurgical-robot power mechanism takes the drive motors 1 as a core, the drive motors 1 are connected to the motor turntables 2 via the output shafts, and then the motor turntables 2 are assembled and connected to the connecting turntables 4 through the intermediate turntables 3 or directly. Such multi-level connection method enables more flexible and accurate power transmission, and is adapted to requirements for micro-scale displacement and multi-degree-of-freedom control of a microsurgical robot in surgical operations. Configuration of multiple drive motors can achieve multi-directional and multi-dimensional motion control, so as to meet complicated operation requirements of microsurgery.

The design of the limiting support bracket 5 plays a critical role in this technical solution, and particularly through cooperation of the limiting protrusions 41 with the stoppers, the connecting turntables 4 are effectively prevented from further rotating when reaching a specific angle. This stopper mechanism not only safeguards the drive motors 1 and the transmission mechanism, but also ensures that the robot will not misoperate due to excessive rotation during operation. In addition, through rational design of the limiting support bracket 5, the whole power mechanism reduces wear of moving parts while ensuring precision, thereby elevating stability and service life of the system.

Configuration of the engagement determination mechanism 8 further enhances an intelligent level of the power mechanism. The engagement determination mechanism 8 can accurately determine the engagement status of the connecting turntable 4 with the motor turntable 2 or the intermediate turntable 3, which is critical to ensure reliability of power transmission. Through a signal sent by the engagement determination mechanism 8, a control system can monitor the engagement status in real time, and ensure each action to be performed in a controlled state, thereby improving safety and precision of the system.

The output shaft of each drive motor 1 is mounted with an absolute motor encoder, which design ensures that even when the motor is powered off or subjected to external interference, the system can still maintain accurate position information. Application of such encoders enables higher precision and reliability of the power mechanism, and helps improve performance of the microsurgical robot in actual operations.

Setting of origin initialization using the above power mechanism includes steps of:
assembling the driven member, and identifying an assembling action by the sensor;
starting the drive motor 1, driving the connecting turntable 4 to be meshed with the motor turntable 2, and the engagement determination mechanism 8 confirming that engagement is completed;
further driving the connecting turntable 4 to rotate to an endpoint of the arc-shaped limiting recess 52, and the limiting protrusion 41 contacting and reaching the stopper; and
monitoring a signal of the encoder by the control system, and when position data are stable and a current of the motor exceeds a threshold, recording a current absolute position as an origin, where
a preset travel range of the instrument is smaller than a travel of the stopper, thereby avoiding mechanical abrasion.

A control logic is as follows:
the limiting support bracket 5 maintains a trend of moving towards the motor support bracket 7 through a spring force, so as to ensure self-alignment in a meshing process;
the system compares the data of the encoder with the current of the motor in real time, thereby double verifying a status of the stopper; and
the arc length of the arc-shaped limiting recess 52 is designed to be differentiated according to instrument types, so as to be compatible with needs of multiple surgical scenarios.

Herein, the system can identify the assembling action of the driven member by the sensor, which involves integration and application of sensor technology with robot control system. A core of this process lies in monitoring an assembling status of the driven member (for example, the surgical instrument or other accessory devices) with the robot power mechanism by the sensor in real time, so as to ensure that the system can accurately learn and confirm connection conditions of individual assemblies before executing the surgical operation.

The sensor is mounted in a critical part of the power mechanism, for example, around the connecting turntable 4 or in an assembling region of the driven member. The sensor may be a photoelectric sensor, a magnetic sensor, a pressure sensor, a proximity sensor, or of other types, and specific selection depends on design requirements and operational environments of the system.

These sensors are responsible for detecting presence and position of the driven member. When the driven member starts to approach and attempts to be assembled with the connecting turntable 4, or with the motor turntable 2 through the intermediate turntable 3, the sensor is capable of detecting occurrence of these actions.

When the driven member gradually approaches an assembling position of the power mechanism, the sensor will capture its corresponding signal, such as a change in distance, a change in pressure or a contact signal. Taking the photoelectric sensor as an example, when the driven member enters a detection range of the sensor, the photoelectric sensor will generate a signal based on interruption or reflection of light beam and transmits the signal to the control system.

After the signal is transferred to the control system, the system will identify and record the approaching of the driven member and change in position. The control system determines whether the driven member is properly seated by comparing real-time detection data with preset assembling position information.

Upon receiving the signal from the sensor, the control system will process the data. If it is detected that the driven member has reached a specified position and met assembling conditions, the system will send a confirmation signal. In this case, the system can further operate the drive motor 1 to perform engagement of the connecting turntable 4 with the motor turntable 2 or the intermediate turntable 3, thus completing the assembling.

If the sensor does not detect proper seating of the driven member or the assembling action is abnormal, the system will send a warning signal or halt subsequent operation, thus ensuring safety and accuracy of the assembling process.

The process of identifying the assembling action by the sensor not only improves a degree of automation of the system, but also greatly reduces risks potentially caused by man-made misoperation. In the microsurgical robot, accurate assembling identification is critical, and implementation of this technical solution ensures correct assembling of the surgical instrument, thereby laying a foundation for subsequent high-precision surgical operations.

A setting method for instrument origin initialization is an important function in a microsurgical robot system. This method can ensure that the robot achieves accurate positioning prior to each operation, thereby realizing high-precision surgical operations. The method first assembles the driven member with the motor turntable 2 or the intermediate turntable 3 through the connecting turntable 4. At this stage, the system can identify the assembling action of the driven member by the sensor, thus ensuring smooth progress of the assembly process.

During engagement, the drive motor 1 starts to rotate, so that the connecting turntable 4 is gradually engaged with the motor turntable 2 or the intermediate turntable 3 under an external force. In this case, the engagement determination mechanism 8 plays a critical role, and is capable of accurately identifying the engagement status and transmitting information to the control system, so that the system monitors the whole engagement process in real time.

When the connecting turntable 4 is rotated to a position of the stopper and is blocked from rotating, the control system can determine whether a current shaft position is affected by the stopper by reading an output of the encoder in real time, and at the same time, the control system monitors current data of the motor as auxiliary parameter. When a position signal output by the encoder remains unchanged, it is indicated that the shaft of the drive motor 1 is restricted, and the system can store an absolute position encoding at this instant as the origin, thus completing the setting of the origin initialization.

The origin calibrated by this method can effectively avoid operational errors caused by displacement of the origin during operation. The setting method streamlines an initialization workflow while ensuring precision, and improves use convenience and operational safety of the system.

However, in the microsurgical-robot power mechanism, the origin initialization is closely associated with the configuration of the stopper. Such correlation not only ensures accuracy of system positioning, but also establishes reliability of robot operations. The following is a detailed description of the stopper as the origin and a relationship between the arc length of the arc-shaped limiting recess 52 and the travel of corresponding instrument.

The stopper is to mechanically limit a maximum or minimum range of motion of a moving part (the connecting turntable 4). In the present disclosure, the stopper is provided on the limiting support bracket 5, and when the connecting turntable 4 is rotated to a preset position of the stopper, the limiting protrusion 41 of the turntable comes into contact with one end of the arc-shaped limiting recess 52 and is forced to stop further rotating. This restricted state signifies that the connecting turntable 4 has reached a limit position of the range of motion thereof.

The origin initialization is to set a certain known, repeatedly accessible physical position as zero point or reference point during system startup or reset. For the microsurgical robot, accurate origin setting is of particular importance, because the origin defines a starting reference position of operation of the robot, and all subsequent operations will be performed based on this position.

Therefore, setting the position of the stopper as the origin is quite rational, because the position of the stopper is fixed by a mechanical structure and is not affected by external factors (such as electromagnetic interference and environmental changes). Thus, when the connecting turntable 4 is rotated to the position of the stopper and stopped, its position is determined and stable. The control system can determine the position information at this instant by reading the signal of the encoder, and record the position as initial position (origin) of the system. This method ensures consistency of the position of the origin after each time of initialization, so that the system can maintain high-precision positioning in subsequent operations.

In addition, the arc-shaped limiting recess 52 is a critical structure designed on the limiting support bracket 5, and it cooperates with the limiting protrusion 41 on the connecting turntable 4 for limiting the range of rotation of the turntable. The arc length of the arc-shaped limiting recess 52 directly determines a rotatable angle of the connecting turntable 4, thus restricting the range of motion of the driven member.

In the microsurgical robot, different surgical instruments may require different motion travels so as to be adapted to various fine operational requirements. The arc length of the arc-shaped limiting recess 52 represents a maximum angle of rotation of the connecting turntable 4, i.e., a travel range of corresponding instrument. For example, the longer the arc length is, the larger the angle of rotation of the connecting turntable 4 is, and the larger the corresponding motion travel of the instrument is; conversely, the shorter the arc length is, the smaller the travel of the instrument is.

By adjusting the arc length of the arc-shaped limiting recesses 52, the system can set different motions travels for different types of instruments. The design flexibility enables the microsurgical robot to be adapted to a variety of surgical scenarios, without having to redesign the whole power mechanism. At the same time, stoppers of the arc-shaped limiting recesses 52 also ensure that the instruments will not exceed predetermined ranges, thereby preventing misoperations or mechanical damages caused by over-travel motion. In addition, during actual operation, the maximum travel range of the instrument is controlled by the control system, and the maximum travel limited by the control system is less than the maximum travel of the stopper, i.e., the connecting turntable 4 will not be rotated to the position of the stopper at the other end of the arc-shaped limiting recess 52, thereby reducing physical abrasion or stress accumulation of corresponding structure.

Regarding the control system, the control system plays a central role in the microsurgical-robot power mechanism, and is responsible for orchestrating synergistic collaboration of the sensors, actuators and feedback mechanisms, so as to ensure accuracy and reliability of surgical operations.

A running process of the control system starts from initialization of devices. Upon startup of the system, a central processing unit loads and executes an operating system first, and then sequentially initializes respective hardware modules. In this process, the control system collects data of an initial state by the sensor, such as positions of respective turntables and a connection status of the instrument. Then, the system will run an origin initialization program, drive the motor to rotate the connecting turntable to the position of the stopper, and record the absolute position at this instant by the encoder as the origin of the system.

In practical operation, the control system monitors the data from the sensor in real time, including position signal, engagement determination signal and any possible abnormality of the instrument. When the sensor detects that the instrument has been correctly assembled, the control system accurately calculates a movement trajectory required by respective actuators using a control algorithm according to a preset surgery scheme, and instructs the drive motor to perform operations based on a calculation result. At the same time, the control system will continuously monitor the feedback signal during the execution, so as to ensure that each action is completed within an expected range. If any deviation or abnormality is detected, the system will immediately adjust a control strategy, and even trigger an alarm or suspend operations when necessary, so as to avoid potential risks.

In the whole process, the control system not only needs to manage real-time operation tasks, but also needs to record and analyze operation data. These data not only can be used for adjusting the current operation in real time, but also can be used as a reference for subsequently optimizing the performance of the system. Upon completion of the surgery, the control system will perform safe shutdown according to a set procedure, thus ensuring that all devices are restored to a safe state, and archive relevant operational logs for subsequent analysis and verification.

Finally, it should be noted that the above-mentioned are merely preferred embodiments of the present disclosure, but are not intended to limit the present disclosure. While the detailed description is made to the present disclosure with reference to the preceding embodiments, for those skilled in the art, they still could modify the technical solutions described in various preceding embodiments, or make equivalent substitutions to some of the technical features therein. Any amendments, equivalent replacements, improvements, and so on, made within the spirit and principle of the present disclosure, should be covered within the scope of protection of the present disclosure.

1. A microsurgical-robot power mechanism, comprising drive motors, motor turntables, intermediate turntables and connecting turntables, wherein several drive motors are provided, an output shaft of each of the several drive motors is respectively assembled and connected to one motor turntable; and the motor turntable is assembled and connected to the connecting turntable through the intermediate turntable, or is directly assembled and connected to the connecting turntable;
the several connecting turntables are provided in a limiting support bracket, each of the connecting turntables is provided with a limiting protrusion on a peripheral surface, the limiting support bracket is provided with a stopper, and when rotated to a certain angle, the connecting turntable is in contact with the limiting protrusion, such that rotation of the connecting turntable is blocked;
each of the several connecting turntables is respectively assembled and connected to a driven member, and types of the driven member comprise instrument and sterile adapter; and
the power mechanism is provided with an engagement determination mechanism, and configured to determine an engagement status of the connecting turntable with the motor turntable or the intermediate turntable.

2. The microsurgical-robot power mechanism according to claim 1, wherein an output shaft of each of the drive motors is mounted with an encoder, wherein the encoder is an absolute motor encoder or an incremental encoder.

3. The microsurgical-robot power mechanism according to claim 1, wherein the connecting turntables are disc-shaped members, with one end having several tooth-shaped protrusions in a circular array, and the other end connected to the driven member; the intermediate turntables are cylindrical members with two ends respectively having several tooth-shaped protrusions in a circular array, both ends being connected to the connecting turntable and the motor turntable; the motor turntables are rod-shaped members, with one end having several tooth-shaped protrusions in a circular array, and the other end connected to the drive motor; and
tips of the tooth-shaped protrusions on the motor turntables, the intermediate turntables and the connecting turntables are provided with rounded or chamfered transitions.

4. The microsurgical-robot power mechanism according to claim 1, wherein bodies of the several drive motors are simultaneously fixedly connected to a motor support bracket; the motor turntables, the intermediate turntables, and the connecting turntable are all located in positions between the limiting support bracket and the motor support bracket; and the limiting support bracket and the motor support bracket are provided with a guiding support bracket, wherein relative position of the guiding support bracket and the motor support bracket is fixed; and
the limiting support bracket has a trend of continuously moving towards the motor support bracket under an external force; the engagement determination mechanism is provided between the limiting support bracket and the motor support bracket, and when the connecting turntable is engaged with the motor turntable or the intermediate turntable, the limiting support bracket drives the engagement determination mechanism to move, and the engagement determination mechanism sends a signal, and conversely, no signal is sent.

5. The microsurgical-robot power mechanism according to claim 1, wherein the limiting support bracket is provided with several turntable slots assembled and connected to the connecting turntables, wherein the turntable slots are circular blind slots, a center of circle of the blind slots is provided with a through hole configured to connect the driven member, and the several turntable slots directly communicate with each other or are independent from each other; and
an outer side of each of the turntable slots is provided with an arc-shaped limiting recess mated with the limiting protrusion, wherein the limiting protrusion is inserted into the arc-shaped limiting recess; and the arc-shaped limiting recess communicates with the turntable slot, and has an inside diameter equal to an outside diameter of the turntable slot, and an outer edge profile being arc-shaped.

6. The microsurgical-robot power mechanism according to claim 4, wherein the engagement determination mechanism comprises a press rod, a reset spring and a position sensor, wherein the press rod has one end fitted on the limiting support bracket all the time under an effect of the reset spring, and the other end penetrating through the guiding support bracket to be assembled and connected to the position sensor, and when the connecting turntable is engaged with the motor turntable or the intermediate turntable, the position sensor is triggered, and conversely, the position sensor is not triggered; and the reset spring has one end fixed on the position sensor, and the other end fitted on a shoulder of the press rod.

7. A setting method for instrument origin initialization of the microsurgical-robot power mechanism according to claim 1, comprising steps of:
assembling a driven member with the motor turntable or the intermediate turntable through the connecting turntable, wherein
when a control system identifies an assembling action of the driven member, the connecting turntable is in contact with the motor turntable or the intermediate turntable but is not fully engaged;
then operating, by the control system, the drive motor to rotate, until the connecting turntable is engaged with the motor turntable or the intermediate turntable under an external force;
once the control system identifying engagement of the connecting turntable with the motor turntable or the intermediate turntable, continuing to operate the drive motor to rotate, until the connecting turntable is rotated to a preset stopper on the limiting support bracket and is blocked from rotating;
reading, by the control system, an output of an encoder in real time, wherein when a position signal output by the encoder remains unchanged, the control system determines that a shaft of the drive motor is restricted, and a status in which the connecting turntable is rotated to the preset stopper and is blocked from rotating is identified; and
storing an absolute position encoding at this instant as an origin in the control system, thus completing setting of origin initialization.

8. The setting method for instrument origin initialization of the microsurgical-robot power mechanism according to claim 7, wherein the control system identifies the assembling action of the driven member by a sensor, wherein the sensor is in communication connection with the control system.

9. The setting method for instrument origin initialization of the microsurgical-robot power mechanism according to claim 7, wherein the control system identifies the engagement of the connecting turntable with the motor turntable or the intermediate turntable by the engagement determination mechanism in communication connection with the control system.

10. The setting method for instrument origin initialization of the microsurgical-robot power mechanism according to claim 7, wherein an output shaft of each of the drive motors is mounted with an encoder, wherein the encoder is an absolute motor encoder or an incremental encoder.

11. The setting method for instrument origin initialization of the microsurgical-robot power mechanism according to claim 7, wherein the connecting turntables are disc-shaped members, with one end having several tooth-shaped protrusions in a circular array, and the other end connected to the driven member; the intermediate turntables are cylindrical members with two ends respectively having several tooth-shaped protrusions in a circular array, both ends being connected to the connecting turntable and the motor turntable; the motor turntables are rod-shaped members, with one end having several tooth-shaped protrusions in a circular array, and the other end connected to the drive motor; and tips of the tooth-shaped protrusions on the motor turntables, the intermediate turntables and the connecting turntables are provided with rounded or chamfered transitions.

12. The setting method for instrument origin initialization of the microsurgical-robot power mechanism according to claim 7, wherein bodies of the several drive motors are simultaneously fixedly connected to a motor support bracket; the motor turntables, the intermediate turntables, and the connecting turntable are all located in positions between the limiting support bracket and the motor support bracket; and the limiting support bracket and the motor support bracket are provided with a guiding support bracket, wherein relative position of the guiding support bracket and the motor support bracket is fixed; and
the limiting support bracket has a trend of continuously moving towards the motor support bracket under an external force; the engagement determination mechanism is provided between the limiting support bracket and the motor support bracket, and when the connecting turntable is engaged with the motor turntable or the intermediate turntable, the limiting support bracket drives the engagement determination mechanism to move, and the engagement determination mechanism sends a signal, and conversely, no signal is sent.

13. The setting method for instrument origin initialization of the microsurgical-robot power mechanism according to claim 7, wherein the limiting support bracket is provided with several turntable slots assembled and connected to the connecting turntables, wherein the turntable slots are circular blind slots, a center of circle of the blind slots is provided with a through hole configured to connect the driven member, and the several turntable slots directly communicate with each other or are independent from each other; and
an outer side of each of the turntable slots is provided with an arc-shaped limiting recess mated with the limiting protrusion, wherein the limiting protrusion is inserted into the arc-shaped limiting recess; and the arc-shaped limiting recess communicates with the turntable slot, and has an inside diameter equal to an outside diameter of the turntable slot, and an outer edge profile being arc-shaped.

14. The setting method for instrument origin initialization of the microsurgical-robot power mechanism according to claim 12, wherein the engagement determination mechanism comprises a press rod, a reset spring and a position sensor, wherein the press rod has one end fitted on the limiting support bracket all the time under an effect of the reset spring, and the other end penetrating through the guiding support bracket to be assembled and connected to the position sensor, and when the connecting turntable is engaged with the motor turntable or the intermediate turntable, the position sensor is triggered, and conversely, the position sensor is not triggered; and the reset spring has one end fixed on the position sensor, and the other end fitted on a shoulder of the press rod.
